(19) United States
(12) Patent Application Publication
(10) Pub. No.: US 2016/0139156 A1
(43) Pub. Date: May 19, 2016

(54) APPARATUSES, METHODS, AND SYSTEMS FOR HOME MONITORING OF PHYSIOLOGICAL STATES AND CONDITIONS

(71) Applicant: Welltwigs LLC, Blaine, MN (US)

(72) Inventor: Murtaza Mahammadali Lakdawala, Blaine, MN (US)

(21) Appl. No.: 14/876,880

(22) Filed: Oct. 7, 2015

Related U.S. Application Data
(60) Provisional application No. 62/081,009, filed on Nov. 18, 2014.

(51) Int. Cl. G01N 33/76; G01N 21/78; G01N 21/84

(52) U.S. Cl. CPC G01N 33/76; G01N 21/8483; G01N 21/78; G01N 2333/59; G01N 2021/7759

(57) ABSTRACT

Methods, systems and apparatuses for monitoring a physiological condition of a user. The apparatus includes a housing, a processor, a power source, a receptacle configured to receive an inserted test strip, an imaging unit configured to image the inserted test strip, and a communication unit configured to communicate data received from the imaging unit to a communication network. The apparatus is configured to read color change results produced by more than one different type test strip, and the more than one different type of test strip each produce a different color to indicate the presence of an analyte. The apparatus may include two separate but connectable units: an analyte sensing unit and a base unit. The apparatus may further include a temperature sensing unit which may alternatively connect with the base unit. The apparatus may communicate with a mobile application on a hand held device.

### BACKGROUND

The testing and diagnosis of medical states and conditions at home are popular due to the convenience and reduced cost compared to visiting a doctor's office. Some medical states and conditions have been monitored by individuals in the home environment for many years. However, advances in medical technology allow for expanded and more accurate use of home testing and diagnosis.

One example of a medical state that has long been monitored at home is fertility, for the purpose of either increasing or decreasing the likelihood of an individual becoming pregnant. One traditional methods of fertility monitoring includes tracking the monthly menstrual cycle to determine the most fertile days of the cycle. While this method can have an impact on pregnancy rates, it is ineffective for many women, such as those with irregular cycles, those who ovulate outside of the expected time window, and those whose ovulation dates within their menstrual cycle vary from month to month. It also provides no information about whether or not ovulation actually occurred.

Another method of fertility monitoring involves tracking a woman's basal body temperature (the temperature when fully at rest) to detect ovulation and therefore the woman's most fertile time. This method is based on detecting a small increase in basal body temperature that occurs at the time of ovulation. With this method, a woman typically takes her temperature immediately upon waking each morning, in order for the temperature reading to be as close to her basal body temperature as possible. She then charts the temperatures on graph paper and watches for an increase which may indicate ovulation. However, the temperature increase that occurs at ovulation is small and can be difficult to detect, and often a woman may not recognize the temperature shift until after she enters the high temperature period, which is after ovulation and could be too late to achieve pregnancy. With this method it is difficult to predict the fertile phase in advance, as is preferred for achieving pregnancy. Furthermore, subtle body temperature changes can occur due to causes other than ovulation, such as due to the woman's movement or the temperature of the room, and these changes have no correlation to ovulation. As a result, the temperature graph might appear to show an ovulatory cycle when in fact no ovulation actually occurred, or it occurred at a different time than it would appear from the graph.

Another method of fertility monitoring at home involves assessing the cervical mucus for changes that indicate ovulation. These changes include subtle variations in viscosity and color that are difficult to recognize. The women who are monitoring themselves must make this assessment by touch and observation of the mucus, such that the determination is subjective. It is therefore a difficult method to use with any accuracy. The observation of the cervical mucus can be combined with temperature monitoring to improve the accuracy, in a process called the sympto-thermal method. However, monitoring both the cervical mucus and the temperature together is still difficult and does not solve the problems associated with these methods.

More recently, home tests have become available for detecting the hormonal changes associated with ovulation. For example, the level of luteinizing hormone (LH) rises 24 to 36 hours prior to ovulation, and this rise can be detected in a woman's urine. Various home fertility test kits are commercially available which detect LH in the urine through use of a specific labeled antibody. Typically, a daily urine test is performed by dipping a test stick in urine. The urine flows through the stick and the labeled antibody produces a color change in the presence of the hormone. The presence of a color change may be detected visually by the user, for a positive or negative result. However, the presence of urine in the stick can itself alter the appearance of the test, and the color change can be hard to visualize, making these tests sometimes difficult for users to interpret. Furthermore, only a positive or negative result is possible for these tests, and it is not possible to determine quantitative hormone levels.

In order to determine quantitative hormone levels, systems have been developed which attempt to assess the amount of color change present in such labeled antibody tests. One method of doing this uses a handheld device like a smart phone to take an image of the test result and uses the image processing system of the device to analyze the color change, with the amount of color change corresponding to the concentration of hormone present in the urine. However, such methods may have poor accuracy due factors such as lighting variations as well as the use of a system not designed for this purpose.

Electronic sensors are also available which are specifically designed for use with a particular labeled antibody test. In some cases, the sensor may be used with a test strip which detects only LH, while in other cases the sensor may be used with a test strip which detects two hormones, such as LH and estrogen. These sensors are specific for reading the particular color of the test with which they are designed to be used and cannot be used for detecting other colors.

Another test which is often performed at home is the pregnancy test. Home pregnancy testing kits are available which similarly test for a hormone, human chorionic gonadotropin (HCG), in a women's urine using a specific labeled antibody. Like the LH tests, the HCG test involves dipping a test stick in urine and watching for a color change. The tests results can be interpreted visually by the user by watching for a color change, or the results can be read with a dedicated electronic reader which detects the specific color change and displays the result as positive or negative for pregnancy. As with the fertility monitoring tests, visual inspection for a color change can be confusing and the observations can be interpreted incorrectly. In addition, when pregnancy testing is performed too early, it can result in a false negative result.

### SUMMARY

Embodiments include apparatuses, systems and methods for monitoring a physiological condition of a user. In some embodiments, the apparatus is a hand held apparatus including a housing, a processor, a power source, a receptacle configured to receive an inserted test strip, an imaging unit configured to image the inserted test strip, and a communication unit configured to communicate data received from the imaging unit to a communication network. The apparatus may be configured to read color change results produced by more than one different type test strip, and the more than one different type of test strip may each produce a different color to indicate the presence of an analyte. For example, the more than one different type of test strips may include urine luteinizing hormone detecting test strips and urine human chorionic gonadotropin detecting test strips.

In some embodiments, the imaging unit may be a red greed blue clear (RGBC) sensor. In some embodiments, the apparatus may also include a light source configured to illuminate a test strip when inserted into the receptacle. The apparatus may be configured to automatically identify the type of test strip inserted into the receptacle.

In some embodiments, the apparatus includes a base unit and an analyte sensing unit. The base unit may include the processor and the analyte sensing unit may include the receptacle, the imaging unit, and the colorimetric unit. In some such embodiments, the base unit may also include a connector and the analyte sensing unit may also include a connector, such that the connector of the base unit is connectable to the connector of the analyte sensing unit to communicate sensed data from the analyte sensing unit to the base unit. The connector of the base unit may be connectable and disconnectable from the connector of the analyte sensing unit. The apparatus may further include a temperature sensing unit. The temperature sensing unit may include a housing, a temperature sensing tip, a measurement circuit, a power source, and a connector. The connector of the temperature sensing unit may be connectable to the connector of the base unit to communicated temperature data from the temperature sensing unit to the base unit. The connector of the temperature sensing unit may be connectable and disconnectable from the connector of the base unit. In some embodiments the housing of the analyte sensing unit is connectable to the housing of the base unit to form a first configuration of the apparatus, and the housing of the temperature sensing unit is connectable to the housing of the base unit to form a second configuration of the apparatus.

Other embodiments include systems for monitoring a physiological condition of a user. The system may include a hand held apparatus for monitoring the physiological condition of the user including a housing, a processor, a power source, a receptacle configured to receive an inserted test strip, an imaging unit configured to image the inserted test strip, and a communication unit configured to communicate data received from the imaging unit to a communication network. The apparatus may be configured to read color change results produced by more than one different type test strip, and each more than one different type of test strip may produce a different color to indicate the presence of an analyte. The system may further include a mobile application operable on a portable computing device and configured to receive the data from the base unit and provide an interface with a user. In some embodiments the data may be obtained from a luteinizing hormone test performed during a user's previous menstrual cycle, and the test may be a luteinizing hormone test. In some embodiments, the data may be data obtained from a luteinizing hormone test, such as from a user's current menstrual cycle, and the test may be a human chorionic gonadotropin test.

The mobile application may be configured to instruct a user to perform a test using the apparatus using the user interface. The mobile application may be configured to analyze the data to determine when the user should perform the test and to instruct the user to perform the test at that time.

In some embodiments, the apparatus includes an analyte sensing unit and a base unit. The analyte sensing unit may include the housing, the receptacle configured to receive an inserted test strip, the imaging unit configured to image the inserted test strip, a power source, and a processor. The base unit may include a housing, the processor, the power source, and the communication unit which may be configured to communicate data received from the analyte sensing unit to a communication network. In some embodiments, the apparatus, also includes a temperature sensing unit including a housing, a sensing tip, a measurement circuit, a power source, and a connector. The base unit may also include a connector as well as the analyte sensing unit. The connector of the analyte sensing unit and the connector of the temperature sensing unit may both be configured to interchangeably connect with the connector of the base unit to communicate sensed data to the base unit. In addition, the housing of the analyte sensing unit and the housing of the temperature sensing unit may both be configured to interchangeably connect with the housing of the base unit.

Still other embodiments include methods of monitoring a physical condition of a user. The method may include connecting an analyte sensing unit to a base unit, inserting a test strip into a receptacle of an analyte sensing unit, applying the user's bodily fluid to a sample portion of the test strip to begin a test for an analyte, and viewing a result of the test on a mobile application on a portable computing device. The fluid may be urine and the analyte may be luteinizing hormone. In some embodiments, the method may further include, after performing the above steps, viewing instructions on the mobile application to perform an human chorionic gonadotropin (HCG) test on or after a specified day, then inserting an HCG detecting test strip into the receptacle of the analyte sensing unit, applying urine to a sample area of the HCG detecting test strip to begin a test for HCG, and viewing a result of the HCG test on the mobile application on the portable computing device.

In some embodiments, the method may further include attaching a temperature sensing unit to the base unit, inserting a temperature sensing tip of the temperature sensing unit into a user orifice to measure the user's temperature, and viewing the temperature results on the mobile application on the portable computing device.

### BRIEF DESCRIPTION OF THE DRAWINGS

The following drawings are illustrative of particular embodiments and do not limit the scope of the inventions. The drawings are not necessarily to scale and are intended for use in conjunction with the following detailed description. Embodiments of the inventions will be described with reference to the drawings, in which like numerals may represent like elements.

FIG. 1 is a block diagram of a monitoring system according to various embodiments;

FIG. 2 is a block diagram of a base unit according to various embodiments;

FIG. 3 is a block diagram of an analyte sensing unit according to various embodiments;

FIG. 4 is a block diagram of a temperature sensing unit according to various embodiments;

FIG. 5 is a perspective view of an apparatus including an analyte sensing unit and a base unit according to various embodiments;

FIG. 6 is a perspective view of the apparatus of FIG. 5 with the analyte sensing unit detached from the base unit and the test strip removed from the analyte sensing unit.

FIG. 7 is perspective view of the electrical components of the analyte sensing unit of FIGS. 5 and 6.

FIG. 8 is a perspective view of the interior components of the analyte sensing unit of FIGS. 5 and 6.

FIG. 9 is a partial cutaway perspective view of the analyte sensing unit of FIGS. 5 and 6.

FIG. 10 is a partial cutaway perspective view of the analyte sensing unit of FIGS. 5 and 6.

FIG. 11 is a partial cutaway perspective view of the analyte sensing unit of FIGS. 5 and 6.

FIG. 12 is a perspective view of the apparatus of FIG. 5 with the analyte sensing unit replaced by a temperature sensing unit.

FIG. 13 is a perspective view of the apparatus of FIG. 12 with the temperature sensing unit detached from the base unit.

FIG. 14 is a perspective view of a test strip according to various embodiments.

FIG. 15 is a flow chart of a method of monitoring and notifying a user of peak fertile days according to various embodiments.

FIG. 16 is a flow chart of a method of monitoring and notifying a user of ovulation according to various embodiments.

FIG. 17 is a flow chart of a method of predicting and notifying a user of a future pregnancy testing date according to various embodiments.

FIG. 18 is a flow chart of method of predicting and notifying a user of a future physiological state according to various embodiments.

FIG. 19 is an image of a user interface of a mobile application according to various embodiments.

FIGS. 20-23 are a series of images of a user interface of a mobile application for ovulation testing according to various embodiments.

FIGS. 24-25 are a series of images of a user interface of a mobile application for basal body temperature monitoring according to various embodiments.

### DETAILED DESCRIPTION

The following detailed description is exemplary in nature and is not intended to limit the scope, applicability, or configuration of the inventions. Rather, the following description provides practical illustrations for implementing various exemplary embodiments. Utilizing the teachings provided herein, those skilled in the art may recognize that many of the examples have suitable alternatives that may be utilized. This application claims priority to U.S. Provisional application No. 62/081,009, filed Nov. 18, 2014 and entitled APPARATUS AND A METHOD FOR MONITORING A PHYSIOLOGICAL CONDITION OF A MAMMAL, the full disclosure of which is hereby incorporated by reference.

Various embodiments include apparatuses, methods and systems for monitoring a physiological condition. The apparatus may be used by a user for home monitoring of the user's physiological status for a variety of purposes such as for fertility monitoring and/or for detection of conditions such as pregnancy or infection, for example. Furthermore, the apparatus may communicate with a portable computing device to enable the user better monitor or detect the state or condition through a portable application on the portable computing device.

In some embodiments, the apparatus includes two separate units, a base unit and a sensing unit. The sensing unit may include the electronic components needed to detect and/or measure a user variable related to a physiological state and generate data. The base unit may include the processor to analyze the data generated by the sensing unit.

The base unit and the sensing unit may be configured to allow them to be physically connected and disconnected from each other by the user as needed. In this way, one base unit can be used with more than one sensing unit interchangeably. For example, a first sensing unit, configured to test for a first user variable, may be attached to the base unit and the apparatus may be used to test for the first user variable. The first sensing unit may then be removed from the base unit and replaced with a second sensing unit, configured to test for a second user variable. The apparatus may then be used to test for the second user variable. The base unit can therefore be used with a variety of interchangeable sensing units, increasing the usefulness of the apparatus for testing and monitoring various conditions. For example, different sensing units may be used to monitor or detect different user variables associated with different conditions. Alternatively, different sensing units may detect different user variables which both relate to the same user physiological state or condition, and these variables may be used in combination to better monitor or detect that state or condition.

Examples of user variables which may be detected and/or measured by the apparatus include the user's body temperature, the user's basal body temperature, and the presence or absence or level or an analyte in the user's bodily fluid. Examples of analytes which may be detected and/or measured include hormones, white blood cells, glucose, alcohol, viruses, bacteria, yeast, bilirubin, ketones, protein, urobilinogen, nitrite, leukocyte esterase, hemoglobin, hematocrit, medications, drugs (substances of abuse).

For example, a hormone may be detected and/or measured in a user urine sample, such as luteinizing hormone (LH), estradiol, or follicle stimulating hormone (FSH), for monitoring fertility status (predicting ovulation). Human chorionic gonadotropin hormone (HCG) may be detected in a user urine sample to detect pregnancy. Hormones such as estrogen and/or progesterone may be detected and/or measured, in the urine for example, for fertility and/or pregnancy monitoring.

The presence or amount of white blood cells in a user urine sample may be detected and/or measured for detecting the presence of a urinary tract infection. Other substances which may be detected and/or measured, or tests which may be performed on a user urine sample according to various embodiments include glucose, bilirubin, ketones, specific gravity, blood, pH, protein, urobilinogen, nitrite, medications, drugs, and leukocyte esterase. Various embodiments may be used to detect infections on or in the user's body such as such as vaginal yeast infections, sexually transmitted diseases, strep throat. For example, a test strip on an apparatus may be brought into contact with a potentially infected body surface to test for infection.

Other fluids which may be analyzed include blood (for glucose, hemoglobin, or hematocrit measurements, medications, or substances of abuse, for example), cervical mucous (for fertility monitoring, detection of yeast, or detection of sexually transmitted diseases, for example), breast milk (for alcohol content, for example), and saliva, for example.

In some embodiments, the systems, methods, and apparatuses provide an improved method of monitoring a user's ovulation, particularly for women with irregular cycles or women who do not ovulate during a menstrual cycle, providing accurate and convenient results by analyzing physiological patterns using data detected by the apparatus and/or provided by the user. The system may further analyze temperature patterns to confirm that ovulation actually did occur.

The base unit is configured to communicate with the sensing unit of the apparatus. In some embodiments, the base unit may be physically connected to the sensing unit by a user, and the communication may occur through an electronic connection such as a port. In other embodiments, the base unit and the sensing unit may not be physically connected but may nevertheless communicate by wireless transmission. The base unit may further communicate with a portable computing device. The portable computing device may have a display and a mobile application which may be used as an interface on the display for the user to communicate with and receive information from the apparatus, such as through the use of a mobile application (app). The base unit may communicate with the portable computing device through a wired connection or may communicate wirelessly.

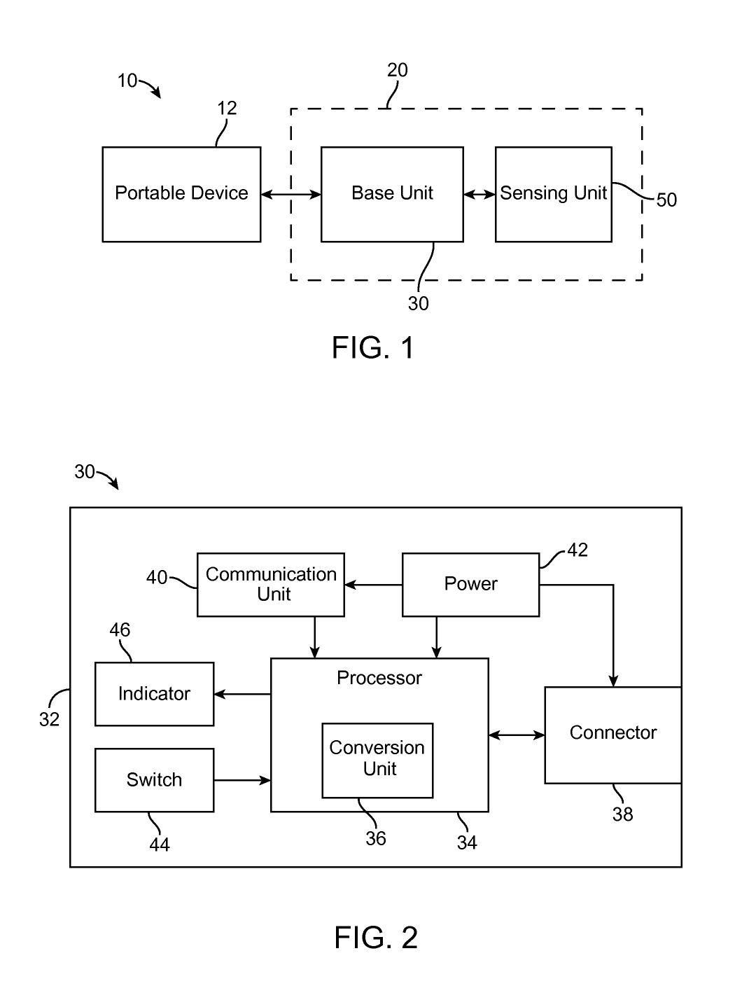

A diagram of a monitoring system 10 according to various embodiments is shown in FIG. 1. The system 10 includes the portable computing device 12 as well as a base unit 30 and the sensing unit 50, which are the two components of the apparatus 20, although the apparatus may also include additional sensing units 50. As indicated by the arrows, the portable computing device 12 and the base unit 30 communicate with each other, and the sensing unit 50 and the base unit 30 communicate with each other. In some embodiments, the base unit 30 may communicate with multiple portable computing devices 12 and with multiple sensing units 50. In some embodiments, the base unit 30 may serve as an independent hardware platform or as a bridge in between the portable computing device or devices 12 and the sensing units 50.

When the apparatus 20 is used as part of a system 10 along with a portable computing device 12, the interface between the user and the portable computing device 12 may provide information to the user, such as results and instructions. It may also allow the user to input data which may be used by the system 10 in combination with the measured variables obtained by the sensing unit 50, for improved detection and monitoring. For example, the user may input information relating to the user's own observations such as menstrual cycle dates (start date, length, and end date, for example), episodes of sexual intercourse, cervical mucous characteristics, basal body temperature, luteinizing hormone test result, pregnancy test result, emotional state, symptoms such as aches and pains, or other information.

Examples of portable computing devices which may be used in various embodiments include smart phones, digital mobile devices, personal digital assistants (PDA's), iPADs, digital display devices, computers (laptops, personal computers (PC's)), tablets, phablets, smart watches, and wearable devices. The portable computing device may be configured with a mobile application. The mobile application may be embedded into any of the mobile operating systems such as android, windows, iOS, Ubuntu, or Java, for example.

An example of a base unit 30 is shown in the block diagram in FIG. 2. The base unit 30 is enclosed within housing 32 and includes a processor 34. The processor 34 processes the signal received from the sensing unit 50 related to the physiological condition of the user. Examples of processors which may be used in various embodiments include digital signal processors (DSP's), microcontrollers, memory units, or any computing unit configured to compute and run the stored algorithms related to the physiological condition of the user as described herein. The processor 34 may be programmed to perform steps for controlling and using various sensing units 50. The processor 34 may have the ability to download applications to manage and control the operation of the sensing units 50.

The processor 34 may include a conversion unit 36. The conversion unit may be a program or formula within the processor 34. The conversion unit 34 may be configured to convert the received digital signal into a pre-defined or standard color model or representation when connected with an analyte sensing unit, such that the processor 34 may compare the value of the digital signal with a pre-stored values of test strip colors when interpreting the results. The color model may be a mathematical model describing the way colors can be represented as tuples of numbers, typically as three or four values or color components. For example, the light sensor may report color values in a first color model such as an rgb color model and the conversion unit may then convert these to a second color model such as HSV and HSL color representations, or CMYK color models.

The base unit 30 further includes a connector 38. The connector 38 provides a physical connection for communication between the two units. In some embodiments, the connector 38 is a port, such as a reconfigurable port, which uses the same input output channels in different hardware configurations. In other embodiments, the connector 38 may be a circular or rectangular connector with multiple electrical contacts. Examples of electrical connectors include various types of USB, hdmi, Apple lightning, aux, and other connectors. The connector may be of a male or female type where in the male type of connector is mounted on the base and the female type of connector is mounted on the sensor, or vice versa.

The base unit 30 further includes a communication unit 40. The communication unit 40 may transmit the sensed information related to the physiological condition of the user over a communication network. The communication unit 40 may transmit the information to the portable computing device 12. The communication unit 40 may transmit the sensed information over a wired network or a wireless network. Examples of wired connections which may be used include USB's, serial over USB, I2C over USB, and electric wire, though other wired networks may be used. Alternatively, the communication unit 40 may provide wireless communication with the portable computing device 12, such as by Wi-Fi, Bluetooth, ZigBee, NFC, and ANT or other wireless communication methods. For wireless communication, the communication unit 40 may include an integrated circuit combined with discreet components including but not limited to antenna, tuning components, and other components.

The base unit 30 of FIG. 2 further includes a power source 34. The power source may provide power to the powered components of the base unit 30, such as the processor 34, the connector 38, and the communication unit 40. In some embodiments, the power source 34 may also provide power to the sensing unit 50 when it is connected to the base unit 30. Examples of power sources include energy storage devices such as batteries like lithium-ion batteries, and fuel cells. In some embodiments, the power source is rechargable.

The base unit 30 of FIG. 2 further includes one or more switches 44. The switch 44 may be used by the user to activate the base unit 30 and/or to activate the sensing unit 50 to perform a test to sense changes in the physiological condition of the user. Examples of switches 44 which may be used in various embodiments include buttons, on/off switches, electronic switches, digital switches, and touch switches, among others.

The base unit 30 may also include a display 46. The display 46 may display information which may be information for the user, such as information related to the physiological condition of the user (such as a positive or negative result or the value of a measured variable), instructions to the user related to steps to perform while using the apparatus 20, or information about that status of the apparatus 20. In alternative embodiments, the apparatus 20 may not include a display 46, or the display 46 may be located on the sensing unit 50, or the display 46 may be separate from the apparatus 20. Examples of displays 46 which may be used in various embodiments include LED displays, LCD displays, OLED displays, TV's, graphic displays, digital displays, buzzers, or any electronic devices configured to display information.

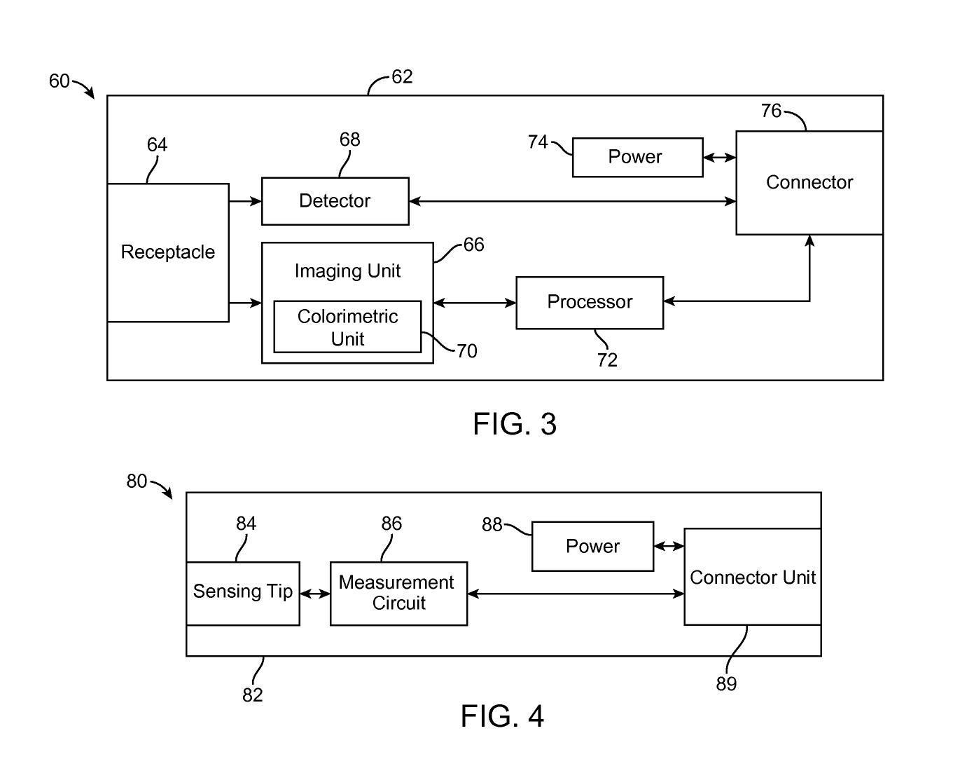

As described above, different sensing units 50 may be used with the base unit 30, depending upon the type of variable being detected and/or measured by the apparatus 20. When the apparatus 20 is used to detect the presence and/or level of an analyte in a fluid, the sensing unit 50 may be an analyte sensing unit, such as the analyte sensing unit 60 depicted in the block diagram shown in FIG. 3.

The analyte sensing unit 60 of FIG. 3 is contained within a housing 62. An aperture within the housing 62 provides access for the fluid into the unit 60 through a receptacle 64. The receptacle 64 receives the fluid, which may be inserted into the receptacle 64 on a carrier such as an absorbent test strip. The test strip may include reagents which may react with the fluid to produce a color change when the analyte is present, and this color change may be detected and/or measured by the sensing analyte sensing unit 60. In this example, the analyte sensing unit 60 includes an imaging unit 66 which may scan the color of the measurement area of the test strip. For example, the analyte sensing unit 60 may detect the presence or absence of lines of different colors. Examples of imaging units 66 which may be used include laser scanners, optical sensors, photo diodes, RGBC sensors, digital camera module and infrared sensors, among others. For example, in some embodiments the imaging unit 66 is an RGBC sensor to read the color of the test and control lines of the test strip and optionally to read any test strip identification mark which may be an identifying color. Such RGBC sensors may provide red, green, blue and clear light sensing for precise color measurement, determination, and discrimination of the test strip, with or without illumination. In some embodiments, the analyte sensing unit 60 may use reflectance or refraction photo colorimetry to read the test strip. Further, the analyte sensing unit 60 may read different colors present on different test strips without the need of recalibration between readings.

The imaging unit 66 may include a colorimetric unit 70. Alternatively, the imaging unit 66 may transmit the scanned color information to a separate colorimetric unit 70. The colorimetric unit 70 may generate an analog signal based upon computing the color of the scanned measurement area of the test strip.

The analyte sensing unit 60 further includes a processor 72. The processor 72 may receive the analog signal from the colorimetric unit 70 and convert it to a digital signal. The processor 72 may be, for example, an analog to digital converter (ADC) or a digital to analog converter (DAC).

The analyte sensing unit 50 may use a color model for precise color measurement, such as the kelvin, HSV (hue, saturation and value), HSL (hue, saturation and lightness), CIE (International Commission on Illumination), or CMYK (cyan, magenta, yellow and key) color models, among others. For example, some embodiments may use HSL as the color model, in which the hue gives the color of the measurement area of the test strip. The saturation of the measurement area of the test strip represents a particular level of the physiological parameter measured depending on the test being used. For example, a pregnancy test may be used in which the color of the measurement area of the test strip is purple. On the HSL model, this may represent a hue value of 0.83. At constant luminance, saturation may be used to identify different shades of the color from light to dark. Another example is a white blood cell test which detects the presence of white blood cells in urine, in which a spectrum of saturation levels of pink represents the amount of white blood cells in the urine, from lighter shade for lesser amounts of white blood cells to a darker shad of pink for greater amounts of white blood cells. Therefore by measuring the saturation value of the measurement area of the test strip the apparatus detects the levels of analyte in the fluid, such as the level of white blood cells in the urine.

Values may be programmed into the system 10, such as threshold values and lookup tables, for use in interpreting the color values, depending upon which color model is used. These values may identify a positive result, negative result, or a quantitative amount. For example, hue and saturation threshold values (for the HSL model), corresponding to a positive or negative result cutoff, may be programmed into the system 10 for each physiological variable tested by the apparatus 20. Similarly, a lookup table of hue and saturation values corresponding to the biological level, such as a concentration level of each analyte, tested by the apparatus 20 may likewise be programmed into the system 10. Such values may be used by the system 10 for each type of test strip and may be may be stored on the portable computing device 12 and/or on the base unit 30, for example. For example, when a test is performed by a user resulting in a color change of the test strip, the measurement of the hue value by the imaging unit 66 is compared to the threshold value or lookup table, and the result may be presented as positive, negative, or a quantitative value.

In some embodiments, the system 10 may correct for any variation in the measured results due to underlying coloration of the test strip, for example. In some embodiments, the imaging unit 66 may measure the color of the measurement area of the test strip prior to exposure to the user's fluid and store this measurement as a reference value. The fluid may then be applied to the test strip, exposing the test strip allowing it to react with any analyte present in the fluid. The imaging unit 66 may then measure the color of the measurement area again to obtain a measured value. The reference value may be subtracted from the measured value to obtain a corrected value, and this corrected value may be used as the user's value for comparison to the programmed threshold and lookup values. Alternatively, the color of one measurement area of the test strip may be read by one imaging unit and may be used as a reference value for correcting results read by another imaging unit in another measurement area.

In some embodiments, the analyte sensing unit 60 also may include a mechanical lever to eject a used test strip out of the receptacle 64.

In some embodiments, the analyte sensing unit also may include a focusing lens. The focusing lens may focus the view of the imaging unit 66 on the measurement area of the test strip for greater precision.

In some embodiments, the analyte sensing unit 60 may include a light source. The light source may illuminate the measurement area or areas of the test strip to improve the readability of the measurement area or areas by the analyte sensing unit 60. Examples of light sources which may be used include LED lights and bulbs.

In some embodiments, the analyte sensing unit 60 may include a black window. The black window ensures that only the measurement area of the test strip is incident on the sensing unit. Furthermore, the black window may partition each measurement area such that each area is visible only to a single sensing unit. The black window may minimize or prevent ambient light from entering the measurement area and affecting the results by maintaining a consistent lighting of the measurement area of the test strip at all times. Furthermore, the black window may minimize or prevent light from a first measurement area of a test strip from affecting a second measurement area of the same test strip.

The black window may be configured such that the measurement areas of the test strip face the imaging units 66 within a chamber of the black window once the test strip has been inserted. In some embodiments, a test strip may include a test line and a control line. The black window may ensure that the test line is only visible to one imaging unit 66 and the control line is only visible to another imaging unit 66. In this way, the black window prevents the colors of different measurement areas, such as a control line and a test line, from interfering with the color measurement of each area.

The analyte sensing unit 60 also may include a detector 68 as in FIG. 3. The detector 68 may automatically detect when a test strip has been inserted into the receptacle 64, such as by scanning the test strip for color changes in the clinical test strip identification area. The detector 68 may also identify the type of test strip being used. In some embodiments, the detector 68 may measure the resistance of the test strip. Other examples of detectors include optical sensors, proximity sensors, and electrical conductivity sensors, for example.

In some embodiments, the apparatus 10 may automatically start the test when the test strip is dipped in or contacted with fluid. For example, when the test strip comes in contact with the fluid, the fluid automatically permeates through the test strip. The fluid comes into contact with the reagents within the test strip, and the presence of the fluid and/or the analyte in the fluid may cause a color change in the reagents, such as due to a reaction between the fluid and/or analyte and the reagents separate and distinct from the test reaction used to measure/detect the target analyte. This color change may or may not be permanent and may only appear momentarily when the fluid first comes into contact with the test strip. This color change may be detected by the sensing unit 60 and this detection may trigger the start of the test by the sensing unit 60. In other embodiments, the sensing unit 60 may detect a change in electrical impedance that occurs when the test strip comes into contact with the fluid, and this detection may trigger the start of the test by the sensing unit. For example, the sensing unit 60 may include conductive contacts configured to contact the test strip after the test strip is inserted into the sensing unit 60. These contacts may be connected to an electrical circuit formed of electrical components that are driven by a controlled voltage or current source. A change in impedance of the test strip after contacting a fluid may result in a change in voltage or current, for example, depending on the circuit design. This change may be measured by an analog to digital converter unit (ADC) in the sensing unit 60 or the base unit 30. The digital value of the impedance measured by this impedance measurement circuit may be used to detect the presence of fluid and thereby trigger start of the test.

The analyte sensing unit 60 may also include a power source 74 which may supply power to all of the sub units. Alternatively, the analyte sensing unit may be powered by the power source 42 in the base unit 30. The analyte sensing unit 60 may include a connector 76 for communication with the base unit 30. The connector 76 may provide communication over either a wired or wireless network as described above with regard to the base unit 30. In some embodiments, the connector 76 is a port to provide a wired connection.

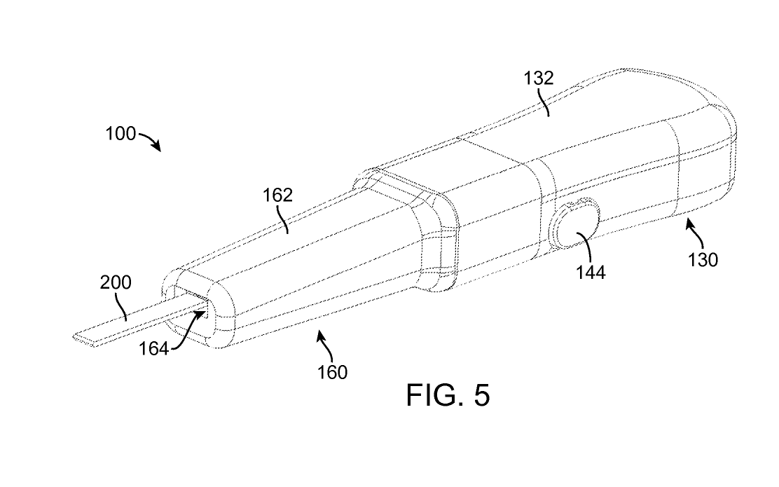

When the sensing unit 50 of the apparatus is used to measure temperature, the sensing unit 50 may be a temperature sensing unit such as the temperature sensing unit 80 shown in the block diagram in FIG. 4. The temperature sensing unit 80 is surrounded by housing 82 and includes a sensing tip 84 which may be used to detect the temperature of a user. The sensing tip 84 may be any shape as appropriate for its location of use on or within the user's body. For example, the sensing tip 84 may be a short funnel shape for use in the ear, an elongated funnel or elongated linear shape for use under the tongue, flat for use on the temple, or any other appropriate shape for the required temperature sensing contact with the user's tissue in the location of use. Other locations into which the sensing unit may be used for sensing temperature include the underarm, rectum, and vagina, for example. In order to detect the user's temperature, the temperature sensing tip 84 may include one or more of a thermistor, infrared, contact sensor, thermopile, semiconductor, surface mounted sensor, platinum wire, fluorescent sensor, thermoelectric sensor, and/or heat flux sensor. The temperature sensing unit 80 may automatically detect when the sensing tip 84 is inside of the ear or in place in any other location for temperature measurement and may then automatically sense the user's temperature.

The temperature sensing unit 80 of this example includes a measurement circuit 86. The measurement circuit 86 measures the sensed temperature and transmits the measured temperature to the communication unit 40 in the base unit 30.

The temperature sensing unit 80 in this example also includes a power source 88. The power source 88 may provide power to all of the powered subunits of the temperature sensing unit. Alternatively, the power source 42 of the base unit may be used to power the temperature sensing unit 80.

The temperature sensing unit 80 also includes a connector 89, as described above with regard to the base unit. The connector provides communication with the base unit 30. The analyte sensing unit 60, the connector 89 may provide communication over either a wired or wireless network. In some embodiments, the connector 89 is a port to provide a wired connection.

The sensing unit 50, whether an analyte sensing unit 60, a temperature sensing unit 80, or some other type of sensing unit, may communicate states of the sensing unit 60 and user results to the portable computing device 12 through the base unit 30. Examples of different states and results may include, but are not limited to, test inserted, test started, test complete, and test result values, to name but a few. The sensing unit 50 may communicate positive results, negative results, any values including numerical and non-numerical values, measurements, and percentages, for example. The sensing unit 50 may communicate that a test produced invalid test results. In some embodiments, the apparatus 20 is used in a system 10 including portable computing device 12, such as a smart phone or other mobile device, including a mobile application that displays the different states of the physiological test of the user through a user interface. The user interface may be configured with the mobile application.

The user interface of the mobile application on the portable computing device 12 may combine the results from multiple sensors to monitor and diagnose a clinical condition or a state of a physiological parameter being evaluated. These results may be further combined with information input into the mobile application by the user. For example, the mobile application may combine temperature trend information during the last menstrual cycle, obtained from temperature measurement made using the apparatus, with user input regarding menstruation dates, to estimate future ovulation dates during a later menstrual cycle. Based upon the estimated ovulation date during a previous menstrual cycle, the mobile application may prompt the user when to perform ovulation tests during a narrower window of time thereby preventing the wastage of test strips and saving the user from the inconvenience of running the test when not necessary.

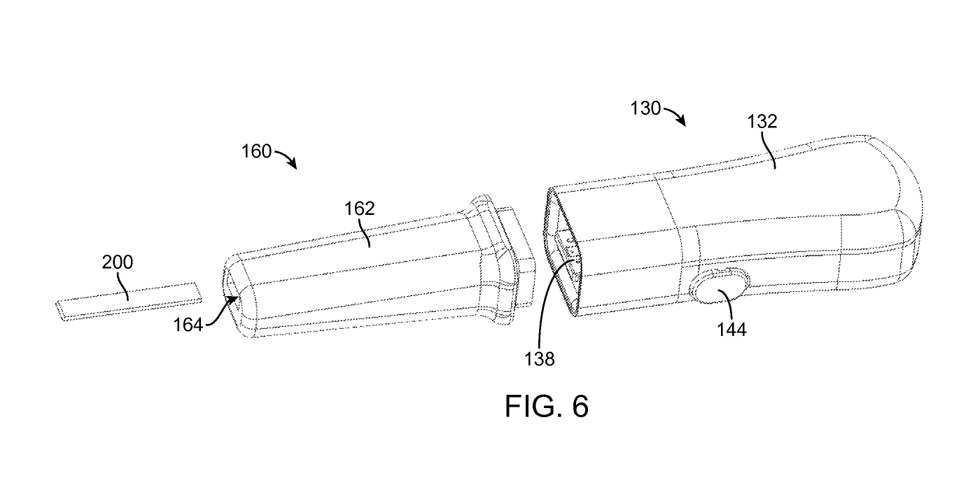

An example of an apparatus according to various embodiments is shown in FIG. 5. The apparatus 100 includes a base unit 130 connected an analyte sensing unit 160. An analyte test strip 200 has been inserted into the receptacle 164 within the housing 162 of the analyte sensing unit 160. The base unit 130 includes a housing 132 and a switch 144. In FIG. 6, the apparatus 100 is shown with the base unit 130 disconnected from the analyte sensing unit 160 and the analyte test strip 200 outside of the receptacle 164.

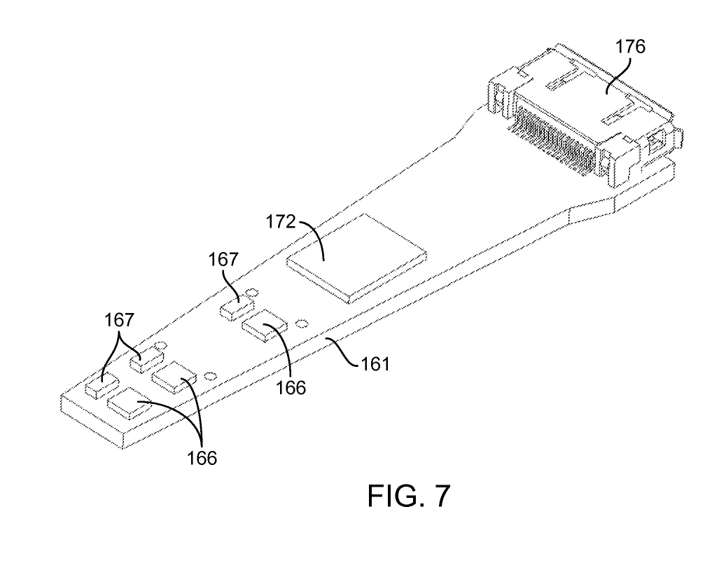

The electrical components of the analyte sensing unit 160 are shown in FIG. 7. A plurality of imaging units 166 are connected to the circuit board 167 near one end of the circuit board 161 and a connector 176 is located at the other end. A plurality of lights sources 167 are also located on the circuit board 167, in proximity to the imaging units 166. The imaging units 166 and light sources 167 are configured within the analyte sensing unit 160 such that, when a test strip is inserted into the receptacle 164, each measurement area is in position to be illuminated by a light source 167 and imaged by an imaging unit 166. A processor 172 is also connected to the circuit board 161.

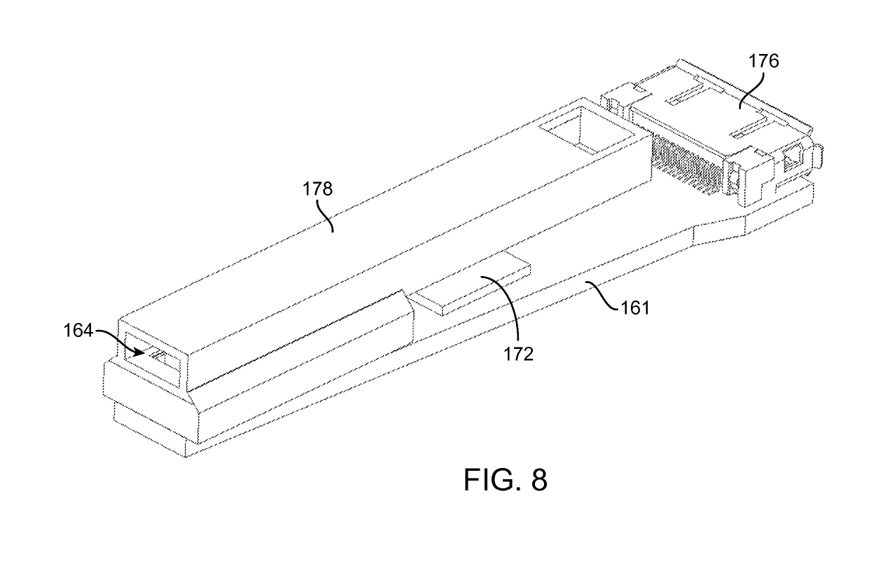

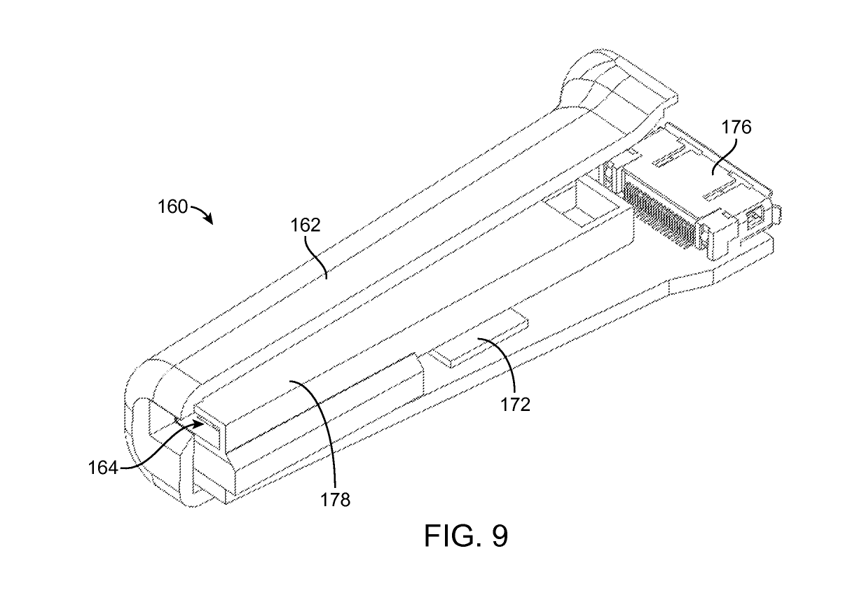

The circuit board 161 of FIG. 7 is shown in FIG. 8, in which the circuit board 161 is covered by a black window 178. The black window 178 includes an aperture which form the receptacle 164 at one end for receiving a test strip.

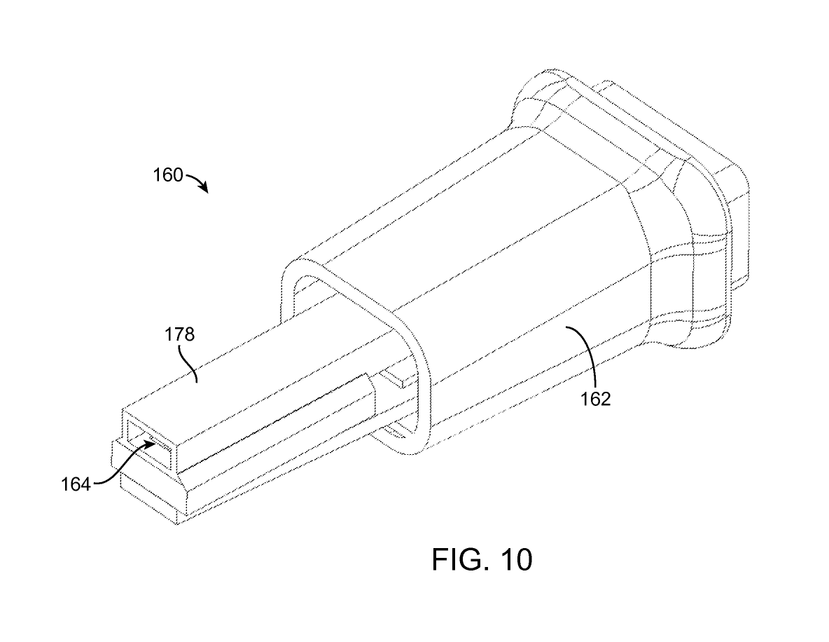

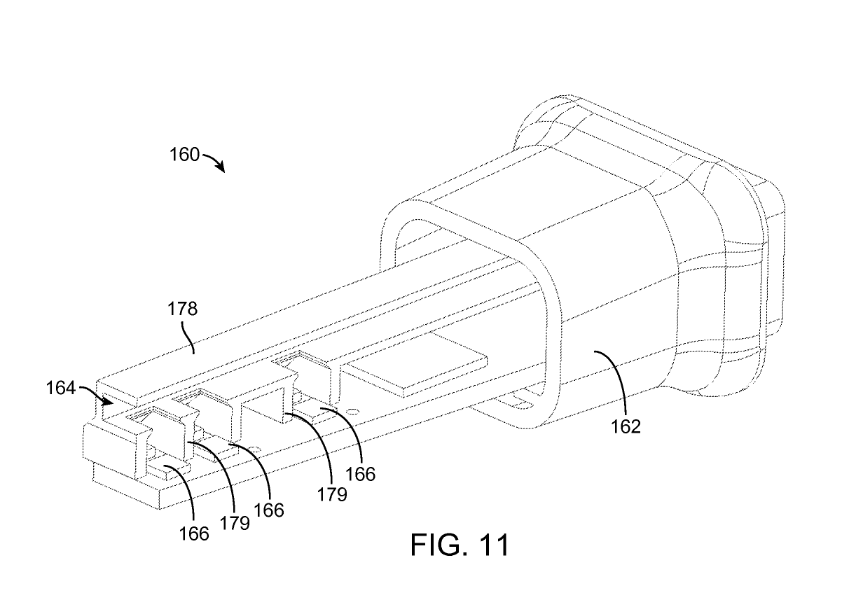

The circuit board 161 and black window 178 of FIG. 8 can be seen inside the analyte sensing unit 160 in FIGS. 9 and 10, in which longitudinal and axial cross sections, respectively, of the housing 162 have been cut away to reveal the internal components of the unit 160. FIG. 11 depicts an axial cross section similar to that shown in FIG. 10 with a further longitudinal cross section through the black window 178 to reveal the partitions 179 that separate the space between each imaging unit. After a test strip is inserted into the receptacle 164, each measurement area (such as each band) will be aligned with an imaging unit 166 and light source 167 within a chamber formed by the partitions 179 in which the only light projecting on the imaging unit is from the adjacent light source 167 within the chamber beneath the test strip.

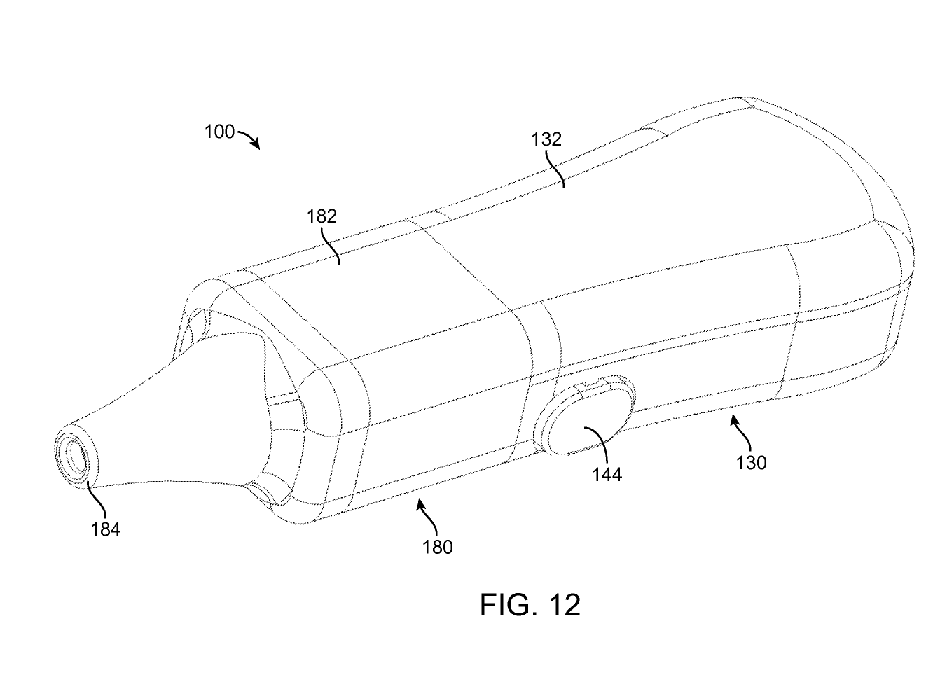

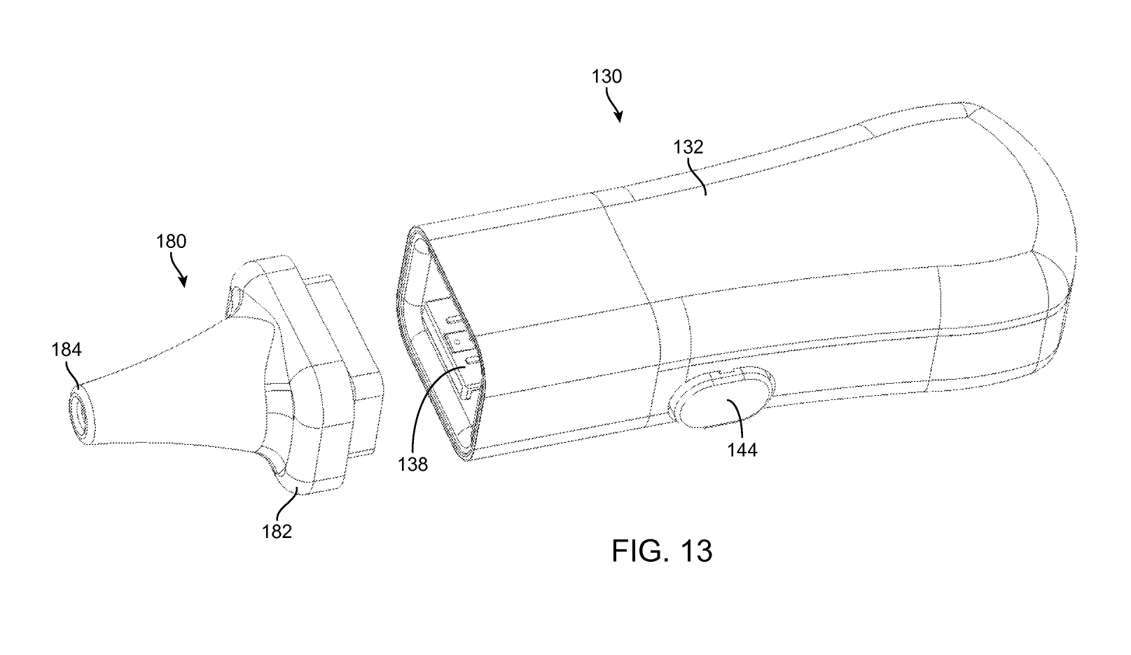

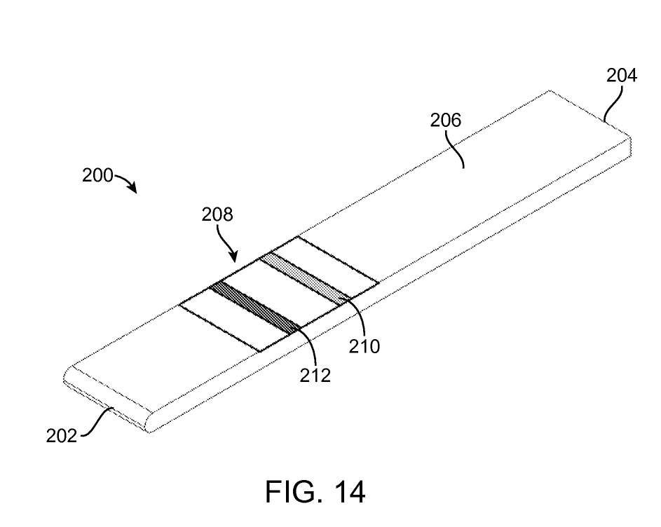

In FIG. 12, the apparatus 100 includes the base unit 130 as in FIGS. 5 and 6 but with an alternative sensing unit which is a temperature sensing unit 180. The temperature sensing unit 180 can be disconnected from the base unit 130, as shown in FIG. 13. The temperature sensing unit 180 includes a housing 182, a sensing tip 184 projecting outward from the unit 180, and a connector 189. The base unit 130 can therefore be used with various sensing units by connecting the connector 138 of the base unit to the connector of the sensing unit. Once assembled together as shown in FIGS. 5 and 12, the two units of the apparatus 100 can fit together smoothly, with the housings and the connectors, interconnecting to form what appears to be, and which functions as, a single unit apparatus which can be easily manipulated by a user using only one hand.

The test strips used in various analyte testing may be clinical membrane strips suitable for lateral flow assays such as lateral flow chromatographic assays, for example. The test strip may include a sample application area at one end, where the user fluid is applied to the test strip such as by dipping the test strip in the fluid. The fluid may automatically flow through the test strip into the measurement area or areas of the test strip. The test strip may include one or more measurement areas including labeled reagents, such as antibodies, to specifically react with an antigen present in the analyte to produce a color change in the measurement area. The assay may be a competitive assay or a sandwich assay, such as an enzyme linked immunosorbent assay (ELISA), for example. Alternatively, the assay may be non-optical such as a magnetic immunoassay (MIA). Fluids which may be analyzed using a test strip include, for example, urine, blood, saliva, cervical mucus, breast milk, or other biological fluids produced by a user.

The test strip may include a single band as a measurement area, which is a test band, in which a color change will occur when the analyte is present. The test strip may optionally include a second band as a second measurement area, which is a control band. The test strip may optionally include additional bands including test bands and control bands. An example of an analyte test strip is shown in FIG. 13. The analyte test strip 200 includes a first end 202 which is inserted into the receptacle 164 of the analyte sensing unit 160, and a second end 204 which projects out of the receptacle and includes the sample application area. After the test strip 200 is inserted into the receptacle 164, the second end 204 may be dipped into the fluid being tested, such as urine, or the fluid may be applied to the sample application on the upper surface 206 of the strip 200 at or near the second end 204. A test portion 208 includes the test band 212 and the control band 210. When the strip 200 is fully inserted into the receptacle 164, the test band 212 aligns with one imaging unit 166 and is lit by one light source 167 and the control band 210 aligns with a second imaging unit 166 and is light by a second light source 167. Additional bands, when present, would be aligned with and imaged by additional imaging units in the same manner.

In some embodiments, the analyte sensing unit 160 is configured to able detect and/or quantify various different analytes. For example, various test strips may be used, each of which detects a different single analyte and produces a different color. In some embodiments, the test strips may detect two or more analytes. Various different test strips can therefore be used with the analyte sensing unit 160 to detect various analytes and various colors.

In order to properly interpret the meaning of the color change (or lack of color change) results that occur for each test strip, the analyte sensing unit 160 must identify the type of test strip which is being used. In some embodiments, the analyte sensing unit 160 may be configured to identify the type of test strip automatically using a characteristic of the test strip. For example, the test strip may include an identifying marking within a measurement area which can be sensed by the imaging unit 166 and used to identify the type of test strip being used, for example based upon the color of the identifying marking, which may be a band of color. Once the apparatus 100 identifies the type of test strip being used, it can automatically apply the correct corresponding test procedures and parameters.

In some embodiments, the identifying mark may be an identification band within the measurement area of the test strip. The identification band may be adjacent to but separate from the test band and the control band (if present). Each type of test strip configured for use with the apparatus may have an identification band of a different color. The imaging unit 166 may image the identification band and identify the color in the same manner that the unit 160 detects and analyzes the test and control bands.

In alternative embodiments, the analyte sensing unit 160 may identify the type of test strip being used by detecting an identification resistor in the test strip. For example, an identification resistor may be embedded on the tip of the test strip. Different identification resistors may be used each having different resistor values, and the analyte sensing unit 160 may detect the resistance value of the identification resistor and use that value to identify the type of test strip being used. For example, the sensing unit 160 may include electrical contacts which contact the test strip after insertion into the sensing unit 160. The electrical contacts may include an identification resistor connected to a measurement circuit on the sensing unit 160 or the base unit 130 that may have a controlled voltage or current source. The identification resistor may detect a change in voltage or current, for example, depending on the circuit design. This change would be measured by an analog to digital converter unit (ADC). The digital value of the resistor measured by this measurement circuit may be used to identify the type of test strip used.

In another embodiment, each test strip may have barcode values that can be read either by the analyte sensing unit 160 or the portable computing device 12. In another embodiment, each test strip may have multiple dots or characters that can be read by the analyte sensing unit 160 to identify the test strip. In such embodiments, the analyte sensing unit 160 may include an imaging component for reading the barcode, dots, or characters, such as a camera.

In still other embodiments, each test strip could include an identification chip. The identification chip may store different parameters for the test in digital format including but not limited to the type of test strip that it is. These parameters can be read by the analyte sensing unit 160 to identify the type of test strip, calibration information about the test strip, the date of manufacturer of the test strip, as well as other information. For example, the sensing unit 160 may include electrical contacts which may be connected to a circuit in the sensing unit 160 or the base unit 130. The processor of the sensing unit may communicate with the identification chip of the test trip via these contacts to read the test strip information mentioned above.

In still other embodiments, an NFC (near field communication) or RFID (radio-frequency identification) tag may be included in the test strip, such as embedded on the test strip. The tag may store different parameters for the test in digital format including but not limited to the type of test it is and the other parameters described above. These parameters can be read by the lab sensing unit by implementing NFC or RFID reader technology which may include a coil or antenna, for example, connected to an integrated circuit that is configured to transmit and receive signals to and from the NFC or RFID tag.

Because the apparatus 20 is capable of detecting and monitoring more than one patient variable, it can aggregate the data regarding the multiple variables in order to provide more accurate analysis. Furthermore, the system 10 is capable of storing data about the user, including the variables measured by the apparatus over time, and this further strengthens the capabilities of the system.

For example, in some embodiments, the apparatus 20 is configured for use in monitoring a user's fertility. The user, in this example a woman, can use the apparatus 20 with the temperature sensing unit 60 to monitor her basal body temperature each day. The system 10 may analyze and track the user's temperature and, based upon the historical temperature values, may predict the most likely time of ovulation. Based upon this prediction, they system 10 may direct the user when to use analyte sensing unit 60 with a hormone detecting strip (such as an LH detecting test strip) in order to detect the hormonal surge that precedes ovulation. Based upon the hormone detection results, the system 10 may provide further information to the user, such as notifying the user when ovulation occurred, notifying the user if ovulation did not occur, and instructing the user when the optimal window of time would be to engage in sexual intercourse in order to achieve conception.

In some embodiments, the system 10 may store a user's data over time to improve diagnostic and predictive ability. For example, when the system 10 is used for fertility monitoring, the system may store the data related to the user's daily basal body temperature measurements, hormone measurements, cervical mucous, menstrual cycle, and/or other data monitored by the apparatus 20 or input by the user. The system 10 may use one or more of these historical data to refine the prediction of the LH surge, and may adjust the instructions to the user accordingly, such as the instructions regarding when to begin hormone monitoring or when to engage in sexual intercourse.

Because the system 10 may be used for testing a variety of variables, and because the apparatus 20 may be used with a variety of sensing units 50, the system 10 may be provided to a user in various combinations. For example, when the initial use of the system 10 is fertility monitoring, the system 10 may be provided to a user as a kit including a base unit 30, an analyte sensing unit 60, a temperature sensing unit 80, and a plurality of hormone detecting test strips for detecting ovulation, such as LH detecting test strips. The kit may further include a mobile application, for use on the user's own portable computing device. The system 10 may also be used for pregnancy testing, with the same analyte sensing unit, but with different test strips. Therefore the kit may further include one or more hormone detecting test strips for detecting pregnancy, or the user may obtain such strips for use with the system 10 at a later time.

The ability to interchange the sensing unit 50 allows the apparatus 20 to be used for detecting and measuring a variety of user variables. That is, while the apparatus 20 may initially be used with one or more sensing units 50 for one purpose, the apparatus 20 may be used for a different purpose later by exchanging the one or more sensing units 50 and/or by using different test strips 200. For example, while a user may initially obtain the system 10 for use in fertility monitoring, the user may later use the system 10 for other purposes, which may be achieved by using different test strips with the analyte sensing unit 60 and/or by using a different type of sensing unit 50 with the apparatus 20. For example, the temperature sensing unit may be used to monitor the temperature of a human for various application like fever, infection, COPD (chronic obstructive pulmonary disease) conditions and other health conditions that require tracking of human body temperature. The analyte sensing unit 60 may be used with different test strips that measure the physiological state of a human body by detecting the state or concentration of an analyte in body fluids like saliva, urine, blood, serum, vaginal secretions, perspiration, breast milk, stool, semen, mucus from nose, ear wax, or other bodily fluids or secretions.

Other types of sensing units 50 may be used as an apparatus 20 with the base unit 30 to measure the state of the user's body. For example, the sensing unit 50 may be an ECG (electrocardiogram) unit configured to measure the electrical activity of the user's heart. Alternatively, the sensing unit may be a stethoscope unit configured to detect and/or measure different body sounds like heart sounds, lung sounds (for example wheezing), abdominal sounds, fetal heart sounds, as well as other types of sounds. In another alternative, the sensing unit 50 may be a fetal heart rate sensing unit which may be configured to measure fetal heart rate using ultrasound Doppler technology. In still other alternatives, the sensing unit 50 may be an imaging unit which may be configured to measure conditions on the surface of the user's body, such as conditions of the skin, such as my measuring the color of the skin. An imaging attachment could be designed that measures the color of the skin thereby detecting skin conditions.

In other cases, the apparatus 20 may be used for a different purpose while using the same sensing units 50 as were used for the original purpose. For example, if the original purpose of the apparatus 20 was fertility monitoring, the same temperature sensing unit 80 as was used for fertility monitoring could also be used for detecting an infection by monitoring temperature. In some cases, the analyte sensing unit 60 used for fertility monitoring could be also used for detecting infection through the use of an appropriate test strip corresponding to the type of infection. For example, if a urinary tract infection is suspected, a test strip may be used which detects the presence of white blood cells in the urine. Similarly, the analyte sensing unit 60 used for monitoring fertility may also be used for detecting pregnancy by using a test strip which detects HCG in the urine, or for detecting alcohol in the breast milk to indicate that the milk should not be used. Furthermore, because the apparatus 20 may be used as part of an integrated system 10, the system 10 can detect ovulation and direct a user when it is time to test for pregnancy, such as through the mobile application on the user's portable computing device 12. This is more accurate than traditional pregnancy detection methods which rely upon the menstrual cycle for determining when to test for pregnancy, and therefore the results are more likely to be accurate.

The process of using the system 10 to monitor fertility begins with a user obtaining the apparatus and downloading the mobile application onto a portable computing device 12. The mobile application may ask the user to input information, such as personal characteristics (age, etc.) start and end date of most recent menstrual cycle, and other user data.

When the users launches the mobile application on a portable computing device 12 such as a smart device like a smart phone, the mobile application may ask the user to enable the wireless connection. For example, in a Bluetooth embodiment, the mobile application may ask the user to enable Bluetooth on the portable computing device 12. Once Bluetooth is enabled, the mobile application may perform discovery of any apparatus 20 of the system 10 in the vicinity. If found, the mobile application may establish a connection with the apparatus 20. If the connection is established and is later lost, the mobile application may request the user to turn on the apparatus 20. In other embodiments, the user may be able to establish a connection between the apparatus 20 of the system 10 and the portable computing device 12 through the apparatus 20, such as by pressing a user interface button on the apparatus 20. Once connection is established between the portable computing device 12 and the apparatus 20, it may be possible to automatically launch the mobile app on the portable computing device 12.

The mobile application and/or the apparatus 20 may send informational or instructional notices to the user regarding how and when to perform the fertility monitoring steps. The user may observe these notices and perform the steps. For example, the notices may instruct the user to check her temperature, to change the sensing unit 50 of the apparatus 20, to check her urine for ovulation (LH) using an ovulation test strip, to engage in sexual intercourse, or to check her urine for pregnancy (HCG) using a pregnancy test strip. The instructions on the mobile application may include details about how to perform each step of each test, for example. The notices may also request the user to input information into the mobile application at particular times, such as the characteristics of the cervical mucus, or the occurrence of sexual intercourse. In response to these instructions, the user may perform the steps as instructed, such as inputting the requested information or using the apparatus to perform the requested tests, and the test results may automatically be sent from the apparatus 20 to the portable computing device 12. The mobile application may aggregate the data and, based upon the aggregated results, may issue further instructions to the user, which the user may then see and then follow.

The mobile application may provide a user interface in which the user can observe the aggregated information, which may include her test results and her input data, over time in various formats. For example, the mobile application may present the aggregated data in a graph over time, such as a graph of temperature and LH levels over time. Alternatively, the mobile application may present the aggregated data in a calendar form, with the important dates and information noted on the calendar, such as the dates of the first day of the menstrual cycle, the occurrence of the temperature increase, the occurrence of the LH spike, sexual intercourse, and cervical mucus characteristics. By presenting this data together, the user can better understand her fertility cycles each month. Furthermore, this aggregated data allows the system 10 to provide better information to the user, such as when to test for pregnancy, and when to seek advice from a physician. For example, if the system 10 observes a certain number of months, such as three months, in which no ovulation occurred, the system 10 may instruct the user to see a physician to assess for a possible medical condition such as polycystic ovarian syndrome (PCOS). The user can then provide the aggregated results to the physician to assist with diagnosis.

Data about the user's physical state may be collected and stored by the system 10 in a data storage unit such as a memory storage unit within the apparatus 20, within the portable computing device 12 and/or in a remote server such as a server in the cloud. This data may be produced by the apparatus 20 through physical measurements and/or the data may be input by the user, such as by inputting the data into a mobile application on a portable computing device 12. For example, the apparatus 20 may transmit the data to the portable computing device 12, which may in turn transmit the data to a server within the cloud for data storage. The system 10 may include instructions for implementation of methods of using this data, and these instructions may be stored on a server, such as a server in the cloud, and/or within the application on the portable computing device 12. User data may be used by the system 10 to improve the capabilities of the system 10 in various ways. For example, in some cases, data about the user's historical physiological state may be used to predict future states and/or to direct user activities at the present or future time. Various examples of the types of data that may be collected and stored and how it may be used are described below. The server may analyze the user data using unique algorithms to calculate different parameters related to a physiological state of a user. By analyzing patterns in the user data, the server based algorithms may be used to diagnose health conditions of the user.

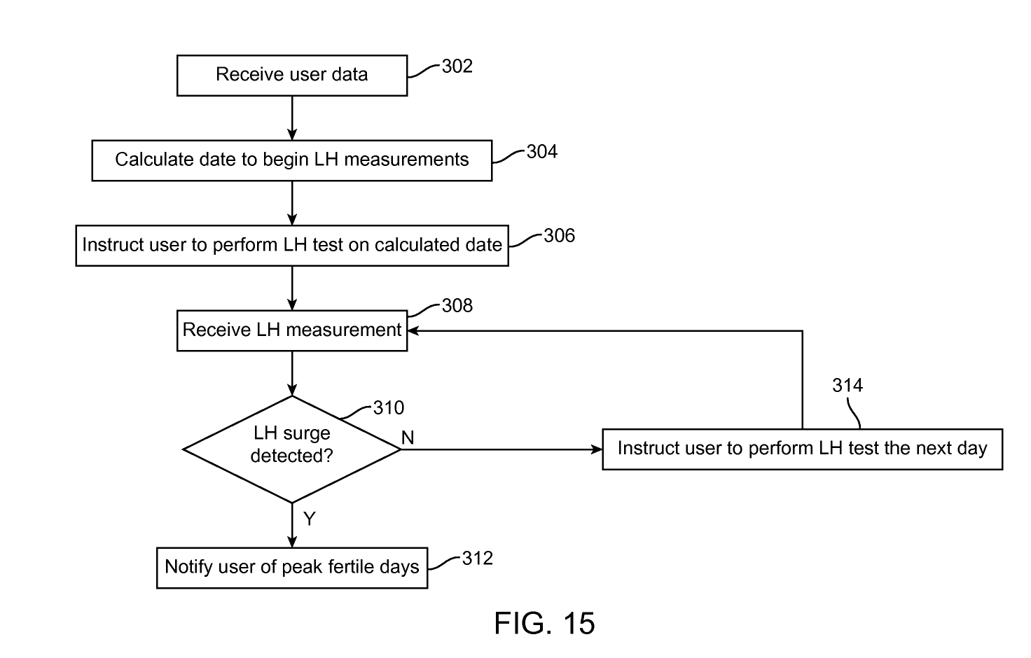

In some embodiments, the system 10 provides a method of detecting hormonal changes to identify a female user's most fertile days. An example of such a method is shown in FIG. 15. The method begins at step 302 when the system receives user data. In this step and in other similar steps of receiving user data in other methods described herein, this data may be input by the user such as through the mobile application or through a website, may be received from the apparatus 20, and/or may be retrieved from storage. For example, the data may include the first day of most recent menstruation. It may also include data regarding ovulation during a previous menstrual cycles, such as the number of days between the first day of menstruation and the LH surge and/or basal body temperature increase. Based upon the user data, the system 10 calculates the date for the user to begin measuring LH (and/or other hormones which correlate with ovulation) in step 304. The system 10 then instructs the user to perform the LH measurement on the calculated date in step 306, such as by sending the user a notification on the mobile application. After the user performs the LH measurement using the apparatus, the system 10 receives the data for the LH measurement in step 308. In step 310 the system 10 determines whether an LH surge is detected, such as by comparing the LH measurement to a threshold value. If an LH surge is detected, the system 10 notifies the user of the user's peak fertile days in step 312. If no LH surge is detected, the system 10 instructs the user to perform the LH measurement the following day in step 314. This cycle repeats until an LH surge is detected. In some embodiments, this cycle may be stopped when a predetermined number of days past predicted ovulation have passed. The cycle may also be stopped when menstruation starts for the user, which clearly indicates that a new menstrual cycle has started and that ovulation was either not detected or never happened in the past cycle. Cervical mucus data may also be input by the user or measured by the device and used as an additional fertility parameter, in conjunction with the LH measurement and the basal body temperature. In some embodiments, cervical mucus data may be logged by the user and may be used to indicate fertile days based on the type of cervical mucus. For example, if the state of cervical mucus is egg white for a given day, the system may indicate to the user that it is a fertile day, while if the cervical mucus is non egg white, the system may indicate to the user that it is a non-fertile day. In some embodiments, LH and basal body temperature data may be given higher priority than cervical mucus data for determining fertility when there conflicting results.

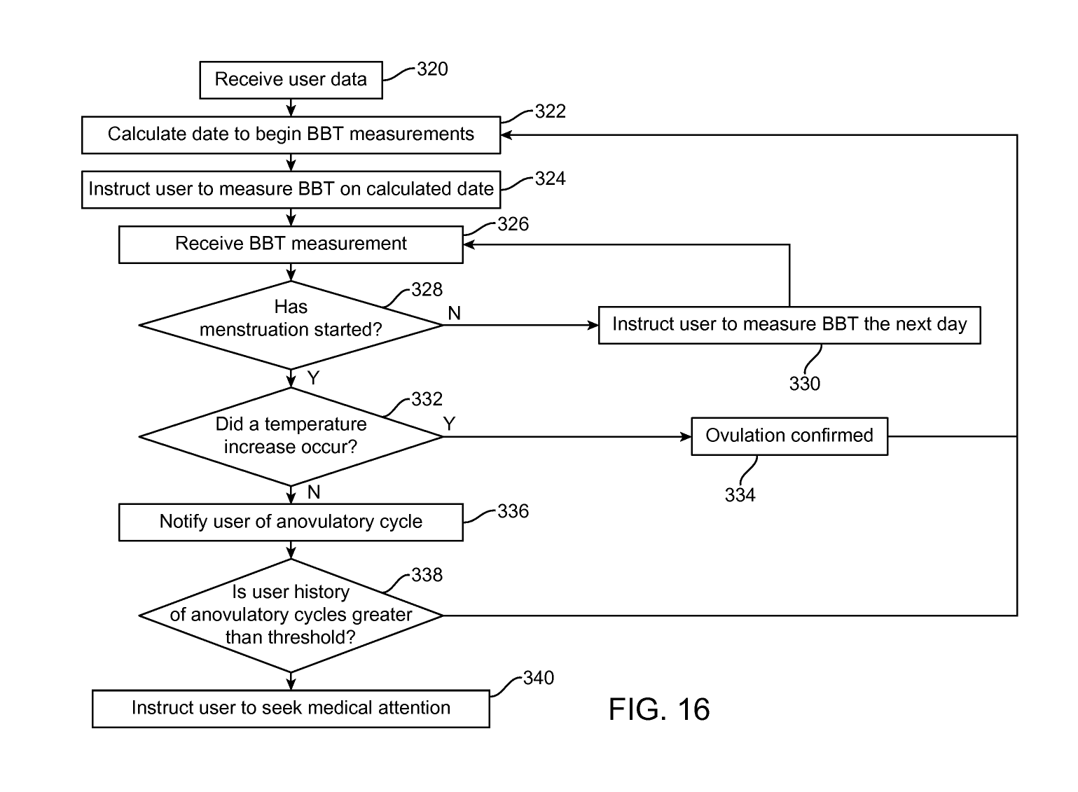

In some embodiments, the system 10 provides a method of confirming that ovulation has occurred using the user's basal body temperature data. An example of such a method is shown in FIG. 16. The method begins with the system 10 receiving user data in step 320. The data may include the first day of the most recent menstruation. It may also include data regarding ovulation during a previous menstrual cycles, such as the number of days between the first day of menstruation and the LH surge and/or basal body temperature increase. In step 322 the system 10 uses this data to calculate the date on which the user should begin measuring basal body temperature. In step 324, the system 10 instructs the user to measure basal body temperature on the calculated date. The user then measures the basal body temperature, such as using the apparatus described herein. The system 10 receives the basal body temperature measurement in step 326. In step 328, the system 10 determines whether menstruation has started based on user input data. If menstruation has not started, the system 10 instructs the user to measure basal body temperature the next day in step 330. The cycle of receiving basal body temperature measurements continues until menstruation starts. Once menstruation starts, the system 10 then evaluates whether a baseline temperature elevation occurred in step 332. The temperature elevation may be identified as an increase in temperature greater than a threshold amount such as 0.2, 0.3, or 0.4., or 0.5 degrees Fahrenheit. For example, the system may identify a temperature elevation in a user by analyzing temperature measurements on successive days. When a group of measurements shifts from low to high values, the system may identify that a temperature elevation occurred consistent with the ovulation. If a temperature elevation is identified, then ovulation is confirmed by the system in step 334 and the user may be notified. The process may then repeat beginning again at step 322. Alternatively, if no temperature elevation is identified, the user may be notified that it was an anovulatory cycle in step 336. In step 338, the system 10 may compare the user's history of anovulatory cycles to a threshold value, such as three consecutive anovulatory cycles. If the user's history of anovulatory cycles is greater than the threshold value, the user may be instructed by the system to seek medical attention. If not, then the process may repeat beginning again at step 322.

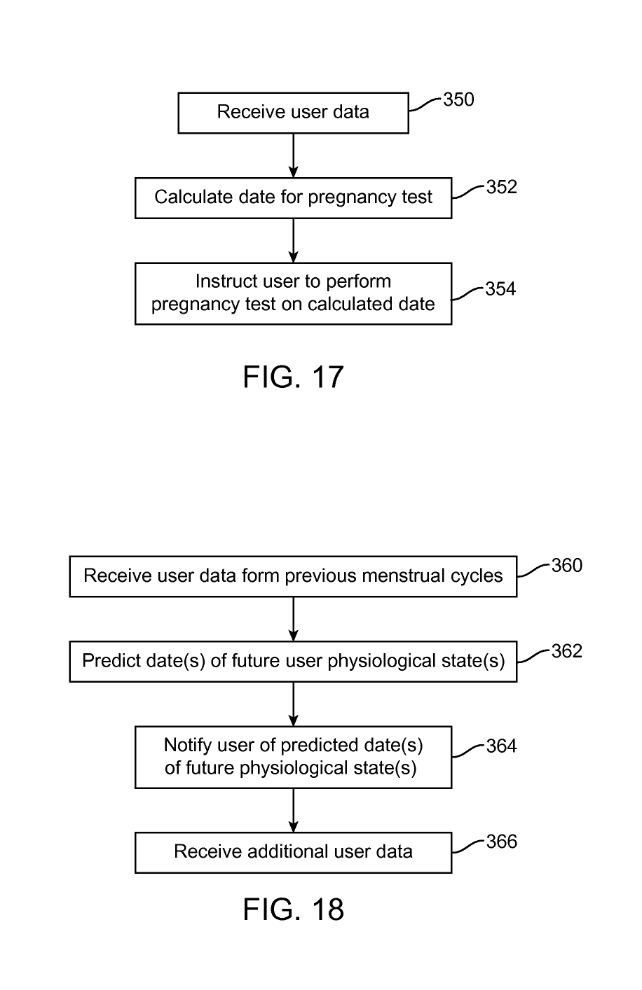

In some embodiments, the system 10 may instruct a user when to perform a pregnancy test based upon user data. An example of such a method is shown in FIG. 17. In step 350, the system 10 may receive user data, such as the date of the first day of the last menstruation, the data of measured hormone changes such as the LH spike, the date of a basal body temperature shift, and/or the dates(s) of intercourse. The system 10 may use this information to calculate a date for performing a pregnancy test in step 352. For example, the system 10 may compare the threshold hormone levels needed to detect pregnancy for the pregnancy test (such as a test strip designed for use with the apparatus 20) to predicted hormone levels for the user on various days based upon the user data, such that the calculated date occurs after the predicted hormone levels of the user are greater than the threshold levels of the test. The system 10 may then instruct the user to perform the pregnancy test on the calculated date in step 354. In this way, the date on which to perform the pregnancy test can be more accurately identified, avoiding the wastage of test strips.

The system 20 may also be used to predict the date(s) of future physiological states of the user as shown in FIG. 18. These future physiological states include ovulation, peak fertility, and menstruation, for example, which can be particularly useful for planning travel for timing of sexual intercourse. In step 360, the system 10 may receive user data from previous menstrual cycles. In step 362, the system 10 may use the data to predict the date(s) of future user physiological state(s). In step 364, the system 10 may notify the user of the predicted date(s) of future physiological states. The system 10 may continue to receive additional user data in step 366, which can be further used to predict date(s) again in step 362. For example, the system 10 may use user data regarding past periods, such as start dates and lengths, to predict future menstruation days. In some embodiments, the system 10 may provide the user with the user's predicted menstruation dates for the next month or two or even the next six months, for example. Similarly, the system 10 may use the user data regarding past ovulation dates and peak fertility dates to calculate predicted ovulation dates and peak fertility dates for the next month or two or the next several months, such as the next 6 months. In this way, the user may plan and adjust travel schedules as needed to maximize the likelihood of achieving pregnancy such as by avoiding separation from the user's sexual partner during the time of peak fertility.

The system 10 described throughout this application includes an apparatus 20 having multiple interchangeable sensing units 50, but other variations of this system 10 are also within the scope of the inventions described herein. For example, various aspects of this invention may alternatively be implemented using one or more stand-alone apparatuses which do not have interchangeable sensing units but rather have one or more dedicated sensing units. For example, various aspects could be used with an analyte sensing apparatus, which may be capable of detecting a single analyte or multiple different analytes by detecting and quantifying different color changes like the analyte sensing unit described herein, and which may communicate with a mobile application on a portable computing device as described herein. Such a dedicated analyte sensing device could be used in combination with a separate dedicated temperature reading device, for example, which may also communicate with the mobile application on the portable computing device 12.

When the apparatus 20 is used as part of a system 10 with a mobile application on a separate portable computing device 12, the user may interact with the apparatus 20 by way of the mobile application. The mobile application may receive results from multiple tests over time, include more than one type of test. The mobile application may prompt users to take different actions, like take a basal body temperature measurement, an ovulation test, a pregnancy test, or log information such as sexual intercourse activity, menstrual cycle information, and cervical mucous evaluation. The application may store the sensor results and the user input information, such as in the cloud, in internet servers, or in a database. The stored data may then be accessed by the user from a different device or devices than the apparatus 20 that was used to take the measurement. Furthermore, the cloud based data can also be shown to a physician to help the physician review the user's physiological data collected by the apparatus to make a clinical decision.

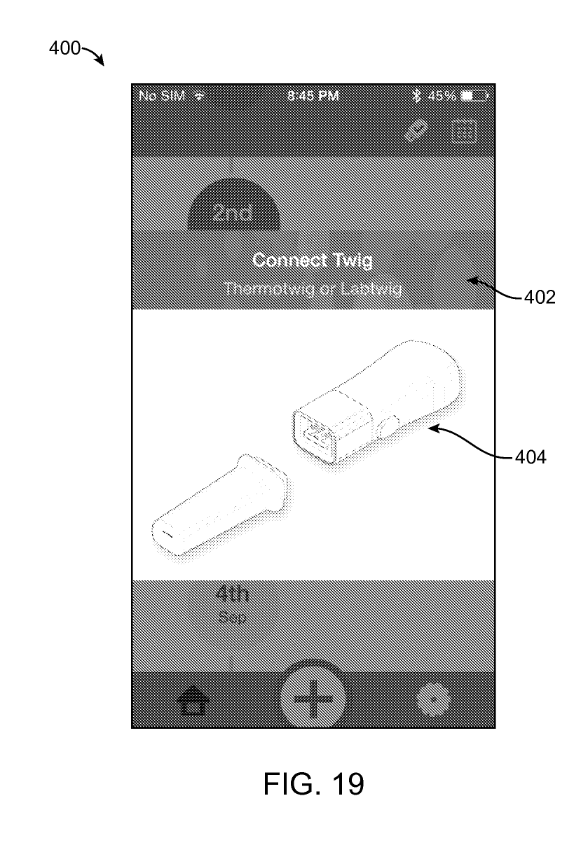

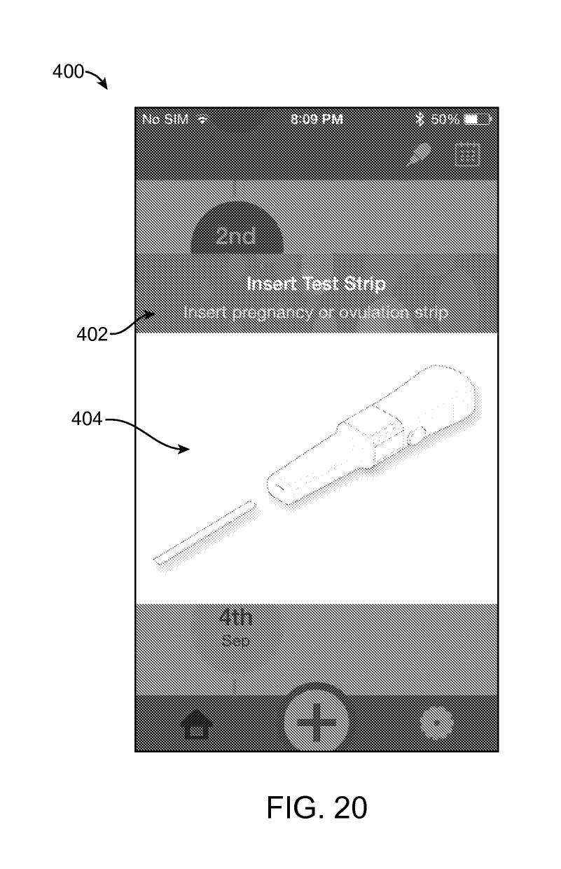

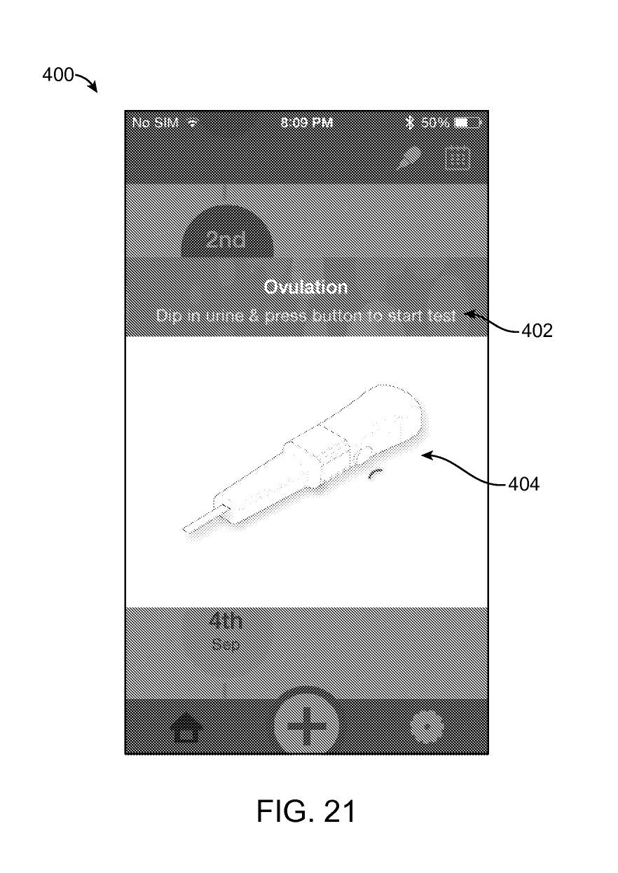

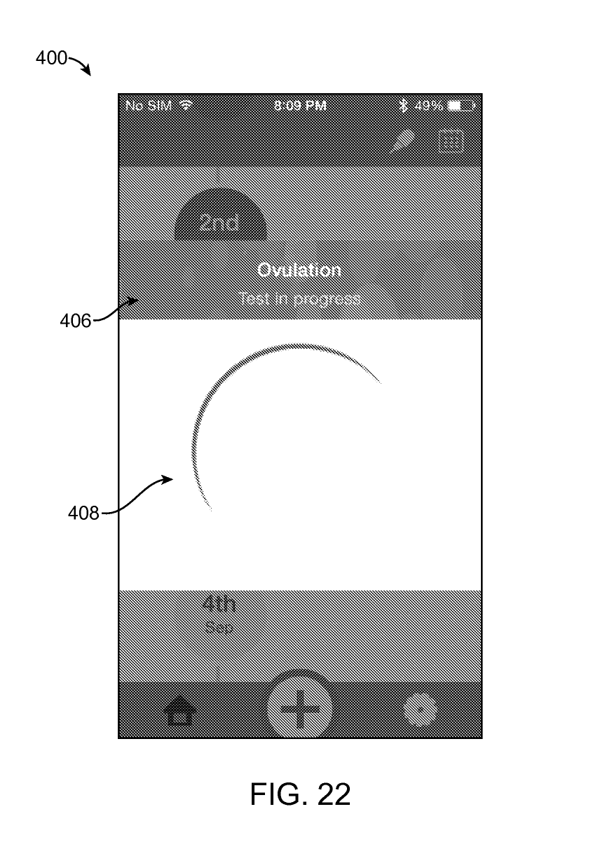

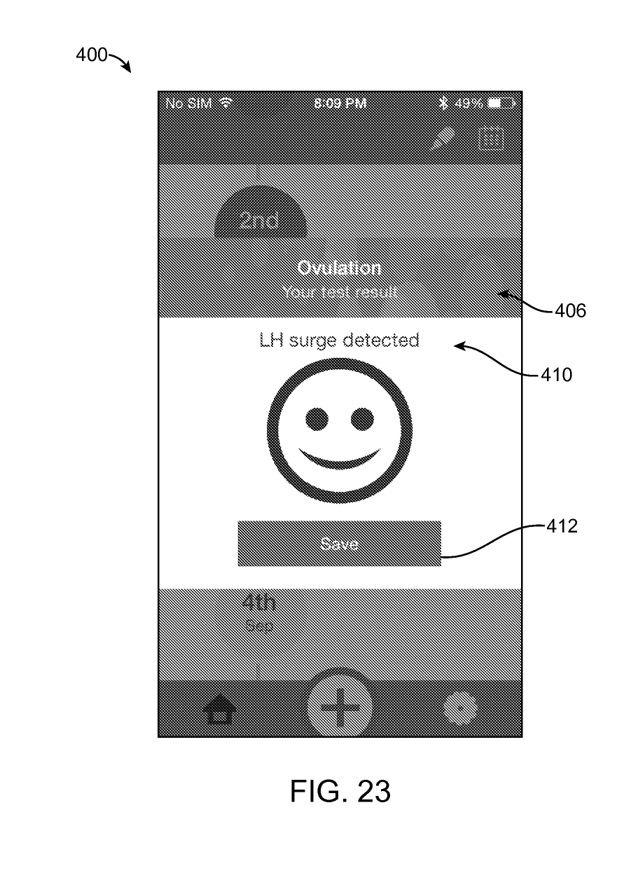

Examples of a user interface for a mobile application are shown in FIGS. 19-25. The interface 400 may include instructions and images depicting the steps to be performed by the user. For example, FIG. 19 includes instructions 402 to a user to connect the base unit to the sensing unit (referred to in this example as a thermotwig or a labtwig) and includes an image 404 of the apparatus with the units in alignment and ready to be connected. The user interface may provide step-by-step instructions as shown in the sequence of user interfaces in FIGS. 20-23, for example. In FIG. 20 the instructions 402 notify the user to insert the test strip and the image 404 shows the corresponding image of an apparatus with a test strip in alignment for insertion into the apparatus. Next in FIG. 21, after the user inserts the test strip and the type of strip is identified by the apparatus, the instructions 402 tell the user the steps for using the test strip, and the image 404 shows the apparatus with the test strip inserted in place. After the user performs the instructed steps, in FIG. 22 the user interface 400 displays a status message 406 indicating the test is in progress and a symbol 408 representing that the test is in progress. Once the test is completed, in FIG. 23 the user interface 400 displays the status message 406, in this case stating that the ovulation test result is being shown, as well as the results message 410, which is that an LH surge was detected. It also provides a button 412 for the user to elect for the system to save the data.

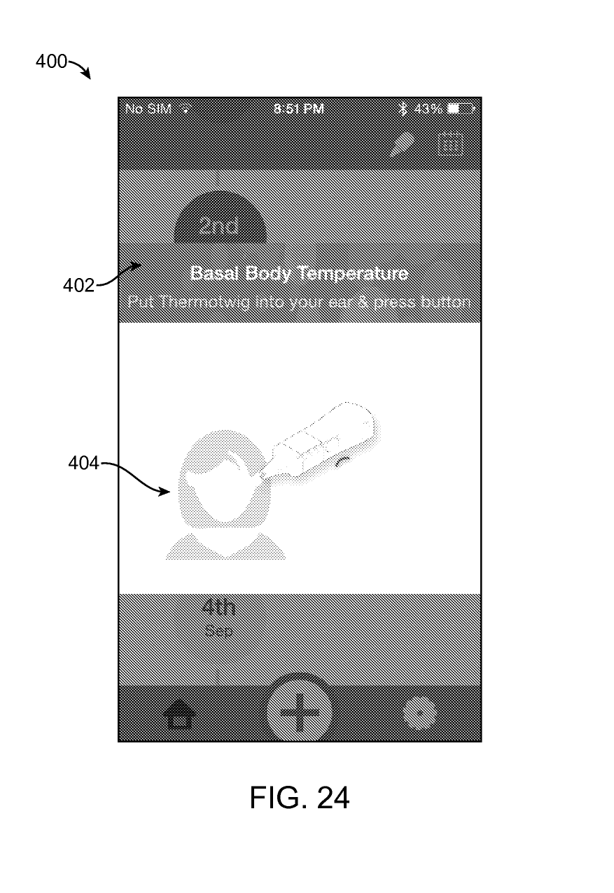

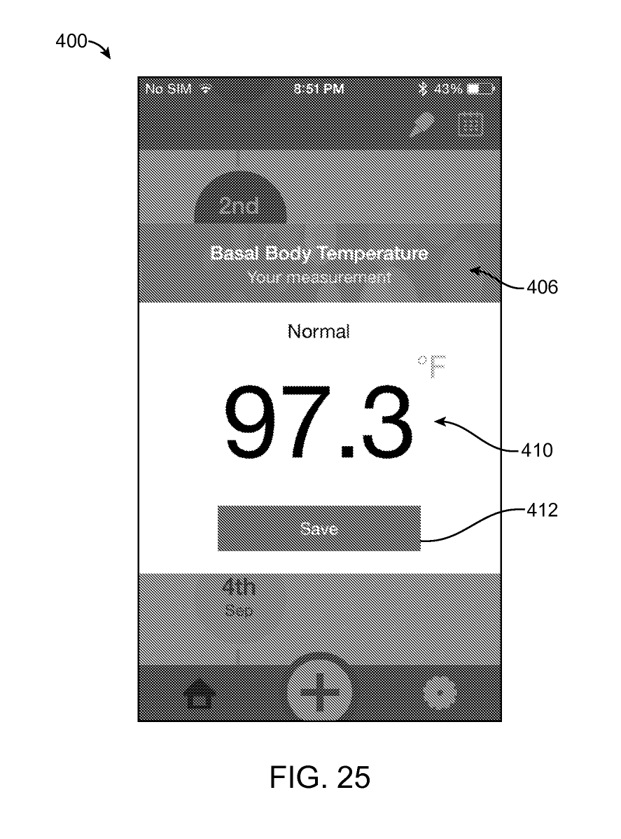

Another sequence of user interface displays are shown in FIGS. 24-25. In FIG. 24, the instructions 402 direct to user how to use the apparatus (the base unit with the temperature sensor) by inserting the apparatus into the ear and pressing start. The image 402 depicts the steps by showing a person with the apparatus in the person's ear. After the user performs the instructed steps, the application displays the status message 406 indicating that the temperature measurement is being shown, as well as the result 410 and the button 412 to save the result.

The system 10 is not limited to fertility monitoring but rather may be used with a wide variety of physical states and conditions. Therefore, depending upon the physiological state or condition being monitored, the system may be used with women or men, including infants, children, and adults. In some cases, the system may be used with non-human animals such as mammals. When the user of the system is a child, infant, or animal, certain steps may be performed by a caregiver rather than the user himself or herself.

In the foregoing description, the inventions have been described with reference to specific embodiments. However, it may be understood that various modifications and changes may be made without departing from the scopes of the inventions.

1. A hand held apparatus for monitoring a physiological condition of a user comprising:
a housing;
a processor;
a power source;
a receptacle configured to receive an inserted test strip;
an imaging unit configured to image the inserted test strip; and
a communication unit configured to communicate data received from the imaging unit to a communication network;
wherein the apparatus is configured to read color change results produced by more than one different type test strip, and
wherein the more than one different type of test strip each produce a different color to indicate the presence of an analyte.

2. The apparatus of claim 1 wherein the imaging unit comprises a red greed blue clear (RGBC) sensor.

3. The apparatus of claim 1 further comprising a light source configured to illuminate the inserted test strip.

4. The apparatus of claim 1 wherein the apparatus is configured to automatically identify the type of test strip inserted into the receptacle.

5. The apparatus of claim 1 wherein the more than one different type of test strips include urine luteinizing hormone detecting test strips and urine human chorionic gonadotropin detecting test strips.

6. The apparatus of claim 1 wherein the apparatus comprises a base unit and an analyte sensing unit, the base unit comprising the processor and the analyte sensing unit comprising the receptacle, the imaging unit, and the colorimetric unit.

7. The apparatus of claim 6 wherein the base unit further comprises a connector, and wherein the analyte sensing unit further comprises a connector, and wherein the connector of the base unit is connectable to the connector of the analyte sensing unit to communicate sensed data from the analyte sensing unit to the base unit, and wherein the connector of the base unit is disconnectable from the connector of the analyte sensing unit.

8. The apparatus of claim 7 further comprising a temperature sensing unit, the temperature sensing unit comprising:
a housing;
a sensing tip;
a measurement circuit;
a power source; and
a connector;
wherein the connector of the temperature sensing unit is connectable to the connector of the base unit to communicated temperature data from the temperature sensing unit to the base unit, and wherein the connector of the temperature sensing unit is disconnectable from the connector of the base unit.

9. The apparatus of claim 8 wherein the housing of the analyte sensing unit is connectable to the housing of the base unit to form a first configuration of the apparatus, and wherein the housing of the temperature sensing unit is connectable to the housing of the base unit to form a second configuration of the apparatus.

10. A system for monitoring a physiological condition of a user comprising:
a hand held apparatus for monitoring a physiological condition of a user comprising:
a housing;
a processor;
a power source;
a receptacle configured to receive an inserted test strip;
an imaging unit configured to image the inserted test strip; and
a communication unit configured to communicate data received from the imaging unit to a communication network;
wherein the apparatus is configured to read color change results produced by more than one different type test strip, and
wherein the more than one different type of test strip each produce a different color to indicate the presence of an analyte, and
a mobile application operable on a portable computing device configured to receive the data from the base unit and provide an interface with a user.

11. The system of claim 10 wherein the mobile application is configured to instruct a user to perform a test using the apparatus using the user interface.

12. The system of claim 11 wherein the mobile application is configured to analyze the data to determine when the user should perform the test, wherein the mobile application instructs the user to perform the test at that time.

13. The system of claim 12 wherein the data comprises data obtained from a luteinizing hormone test performed during a user's previous menstrual cycle, and wherein the test comprises a luteinizing hormone test.

14. The system of claim 12 wherein the data comprises data obtained from a luteinizing hormone test, and wherein the test comprises a human chorionic gonadotropin test.

15. The system of claim 10 wherein the apparatus comprises an analyte sensing unit and a base unit, the analyte sensing unit comprising:
the housing;
the receptacle configured to receive an inserted test strip;
the imaging unit configured to image the inserted test strip;
a power source; and
a processor; and
the base unit comprising:
the housing;
the processor;
the power source;
a communication unit configured to communicate data received from the analyte sensing unit to a communication network.

16. The system of claim 15, wherein the handheld apparatus further comprises:
a temperature sensing unit comprising:
a housing;
a sensing tip;
a measurement circuit;
a power source; and
a connector;
wherein the base unit further comprises a connector;
wherein the analyte sensing unit further comprises a connector;
wherein the connector of the analyte sensing unit and the connector of the temperature sensing unit are both configured to interchangeably connect with the connector of the base unit to communicate sensed data to the base unit; and
wherein the housing of the analyte sensing unit and the housing of the temperature sensing unit are both configured to interchangeably connect with the housing of the base unit.

17. A method of monitoring a physiological condition of a user comprising:
connecting an analyte sensing unit to a base unit;
inserting a test strip into a receptacle of an analyte sensing unit;
applying the user's bodily fluid to a sample portion of the test strip to begin a test for an analyte;
viewing a result of the test on a mobile application on a portable computing device.

18. The method of claim 17 wherein the fluid comprises urine and wherein the analyte comprises luteinizing hormone.

19. The method of claim 18 wherein the method further comprises:
attaching a temperature sensing unit to the base unit;
inserting a temperature sensing tip of the temperature sensing unit into a user orifice to measure the user's temperature;
viewing the temperature results on the mobile application on the portable computing device.

20. The method of claim 17 further comprising:
after completing the steps of claim 17, performing the following steps:
viewing instructions on the mobile application to perform a human chorionic gonadotropin (HCG) test on or after a specified day;
then inserting an HCG detecting test strip into the receptacle of the analyte sensing unit;
applying urine to a sample area of the HCG detecting test strip to begin a test for HCG;
viewing a result of the HCG test on the mobile application on the portable computing device.
